(19) United States
(12) Patent Application Publication
(10) Pub. No.: US 2026/0183732 A1
(43) Pub. Date: Jul. 2, 2026

(54) PROCESS FOR CONVERTING A FEED IN A SERIES OF REACTORS

(71) Applicant: UOP LLC, Rosemont, IL (US)

(72) Inventors: Scott Lyle Nauert, Chicago, IL (US); Phillip F. Daly, Palatine, IL (US); Zudtky Wisecarver, Long Grove, IL (US); Roberto Rodriguez, Chicago, IL (US); Abhijit Bikas Pal, Gurgaon (IN)

(21) Appl. No.: 19/415,442

(22) Filed: Dec. 10, 2025

(30) Foreign Application Priority Data
Dec. 31, 2024 (IN) 202411104936

(51) Int. Cl. B01J 8/18; B01J 8/26; C07C 4/00; C10G 50/00

(52) U.S. Cl. CPC B01J 8/1836; B01J 8/1827; B01J 8/26; C07C 4/00; C10G 50/00; B01J 2208/00203; B01J 2208/00212; C10G 2300/1044; C10G 2400/08; C10G 2400/28

(57) ABSTRACT

A process for converting a feed in a series of reactors is disclosed. The process comprises taking at least two hydrocarbon streams, a first hydrocarbon feed stream and a second hydrocarbon feed stream. The first hydrocarbon feed stream and the second hydrocarbon feed stream are heat exchanged in a heat exchanger with a second product stream from a second reactor. A heat exchanged first hydrocarbon feed stream is charged into a first catalyst bed in a first reactor to produce a first product stream and a heat exchanged second hydrocarbon feed stream is charged into a second catalyst bed in the second reactor to produce the second product stream. The second product stream is at a higher temperature than the first hydrocarbon feed stream and the second hydrocarbon feed stream.

### FIELD

The field is a process for converting a feed in a series of reactors. The field may particularly relate to a process for converting a plurality of feed in a series of reactors.

### BACKGROUND

Oil and gas refiners worldwide are exploring methodologies and routes to reduce the carbon emissions and are moving towards sustainable processes. One of the approaches includes producing fuels from alcohols such as methanol and alcohol. The alcohol can be converted to olefins and the olefins are used to produce sustainable fuel. The methanol to jet fuel process includes many process steps such as methanol synthesis and oligomerization steps which are highly exothermic. An ethanol to jet fuel (ETJ) process is one of the routes that holds promise to minimize or eliminate carbon emissions. The end product of the ETJ process is jet and diesel fuel produced out of ethanol. The jet fuel is a sustainable aviation fuel and is intended to replace jet fuel produced out of conventional sources such as crude oil. The ETJ process includes many exothermic steps that generate significant heat in the process. If not judiciously recovered this heat would be lost.

Light olefin production is vital to the production of sufficient plastics to meet worldwide demand. Dehydrogenation (PDH) is a process in which light paraffins such as ethane and propane can be dehydrogenated to make ethylene and propylene, respectively typically in the presence of a catalyst. Dehydrogenation can be achieved in either the presence of an oxidant such as oxygen or in the absence of an oxidant. Non-oxidative dehydrogenation is an endothermic reaction which requires external heat to drive the reaction to completion. Propane dehydrogenation (PDH) is a widely practiced process of non-oxidative dehydrogenation to produce propylene from propane. Ethane oxidative dehydrogenation is a newer oxidative process for converting ethane to ethylene which can be conducted at lower temperatures with lower carbon oxide emissions than non-oxidative and thermal cracking processes.

Fluid catalytic cracking (FCC) is another endothermic process that can be tuned to produce substantial propylene. However, not every FCC unit is tuned to make substantial propylene. Also, high propylene FCC units do not recover much ethylene; less than 1% of global ethylene supply comes from FCC.

The great bulk of the ethylene consumed in the production of plastics and petrochemicals such as polyethylene is produced by the thermal cracking of hydrocarbons. Steam is usually mixed with the feed stream to the cracking furnace to reduce the hydrocarbon partial pressure and enhance olefin yield and to reduce the formation and deposition of carbonaceous material in the cracking reactors. The process is therefore often referred to as steam cracking or pyrolysis.

Paraffins with a range of carbon numbers can be thermally cracked to produce olefins including ethane, propane, butanes, and naphtha. Ethane and naphtha feeds are typical due to higher light olefin yield than propane and butane feeds. Ethane feed is used in regions where light hydrocarbon gases are prevalent. In regions, where gas is not abundant, naphtha feed is employed for steam cracking. Naphtha steam cracking has long set the price in the ethylene industry due to higher production cost versus ethane steam cracking. The world does not currently produce enough ethane to supply the growing demand for ethylene. Therefore, regions lacking ethane supply such as Asia and Europe rely mainly on naphtha steam cracking to supply ethylene. Naphtha steam cracking yields only about 30%-35% ethylene with the balance including both relatively high-value by-products comprising propylene, butadiene, and butene-1 and relatively low value by-products comprising pyoil, pygas, and fuel gas. Additional pressures on naphtha steam cracking including minimum production requirements and environmental concerns have led to the withholding of government approvals in certain regions such as China.

A heat exchanger is a device whose purpose is to lower or raise the temperature of a fluid by facilitating heat transfer between that fluid with another fluid at a different temperature. There are several types of heat exchangers available that vary in heat transfer capability; geometrical design and complexity; cost; working fluid temperature, pressure, and viscosity ranges; and many other attributes. These heat exchangers have limitations which restrict their use in some conditions. An improved heat exchanger is needed for the industry.

There is a continuing need, therefore, for improved heat management for exothermic processes. Also, there is a need for improved ways of heat management for endothermic processes which can heat the reactor feed using the reactor effluent that is at a higher temperature than the reactor feed.

### BRIEF SUMMARY

A process for converting a feed stream in a series of reactors is disclosed. The process comprises taking at least two hydrocarbon streams, a first hydrocarbon feed stream and a second hydrocarbon feed stream. The first hydrocarbon feed stream and the second hydrocarbon feed stream are heat exchanged in a heat exchanger with a second product stream from a second reactor. A heat exchanged first hydrocarbon feed stream is charged into a first catalyst bed in a first reactor to produce a first product stream and a heat exchanged second hydrocarbon feed stream is charged into a second catalyst bed in the second reactor to produce the second product stream. The second product stream is at a higher temperature than the first hydrocarbon feed stream and the second hydrocarbon feed stream. The process can be selected from any exothermic process producing a product stream having a higher temperature than the feed stream.

### BRIEF DESCRIPTION OF THE DRAWINGS

FIG. 1 is a schematic drawing of a process and apparatus in accordance with an embodiment of the present disclosure.

FIG. 2 is a schematic drawing of a heat exchanger in accordance with an exemplary embodiment of the present disclosure.

### DEFINITIONS

The term “communication” means that fluid flow is operatively permitted between enumerated components, which may be characterized as “fluid communication”.

The term “downstream communication” means that at least a portion of fluid flowing to the subject in downstream communication may operatively flow from the object with which it fluidly communicates.

The term “upstream communication” means that at least a portion of the fluid flowing from the subject in upstream communication may operatively flow to the object with which it fluidly communicates.

The term “direct communication” means that fluid flow from the upstream component enters the downstream component without passing through any other intervening vessel.

The term “indirect communication” means that fluid flow from the upstream component enters the downstream component after passing through an intervening vessel.

As used herein, the term “predominant” or “predominate” means greater than 50%, suitably greater than 75% and preferably greater than 90%.

The term “Cx” is to be understood to refer to molecules having the number of carbon atoms represented by the subscript “x”. Similarly, the term “Cx−” refers to molecules that contain less than or equal to x and preferably x and less carbon atoms. The term “Cx+” refers to molecules with more than or equal to x and preferably x and more carbon atoms.

The term “column” means a distillation column or columns for separating one or more components of different volatilities. Unless otherwise indicated, each column includes a condenser on an overhead of the column to condense and reflux a portion of an overhead stream back to the top of the column and a reboiler at a bottom of the column to vaporize and send a portion of a bottoms stream back to the bottom of the column. Feeds to the columns may be preheated. The top pressure is the pressure of the overhead vapor at the vapor outlet of the column. The bottom temperature is the liquid bottom outlet temperature. Overhead lines and bottoms lines refer to the net lines from the column downstream of any reflux or reboil to the column. Stripper columns may omit a reboiler at a bottom of the column and instead provide heating requirements and separation impetus from a fluidized inert media such as steam. Stripping columns typically feed a top tray and take the main product from the bottom.

As used herein, the term “separator” means a vessel which has an inlet and at least an overhead vapor outlet and a bottoms liquid outlet and may also have an aqueous stream outlet from a boot. A flash drum is a type of separator which may be in downstream communication with a separator that may be operated at higher pressure.

As used herein, the term “T10”, or “T90” means the temperature at which 10 mass percent, or 90 mass percent, as the case may be, respectively, of the sample boils using ASTM D-86 or TBP.

### DETAILED DESCRIPTION

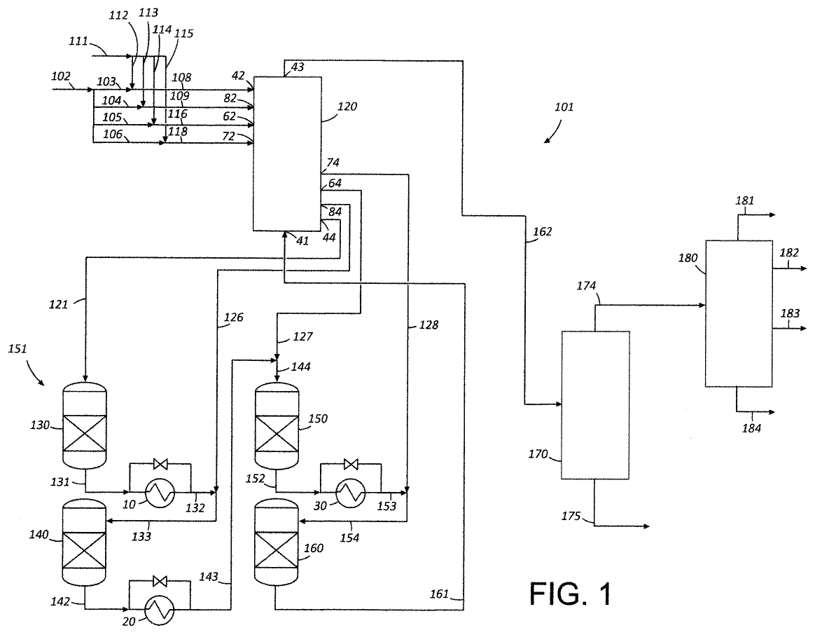

Referring to FIG. 1, an embodiment of a process and an apparatus for converting a feed in a series of reactors 101 is disclosed. In accordance with the present disclosure, the process for converting a feed in a series of reactors may include an exothermic process which produces a product stream having a higher temperature than the feed to the process. In an aspect, the process for converting a feed in a series of reactors may be selected from a methanol to jet process, an ethanol to jet process, and a naphtha to ethane and propane (NEP) process.

The methanol to jet process and the ethanol to jet process comprise various steps such as oligomerization and hydrogenation. These processes comprise producing olefins from alcohol. The olefins can be oligomerized to produce ethylene and propylene to longer chain olefins such as C4 olefins to C20 olefins. The hydrogenation unit hydrogenates olefins to paraffins. The oligomerization and the hydrogenation steps are exothermic and generate heat.

In the NEP process, C3-C8 hydrocarbon feed stock is primarily charged to a “Naphtha to Ethane and Propane” unit to convert naphtha in the presence of hydrogen into desirable ethane and propane along with less desirable methane. “Naphtha to ethane and propane” is a highly exothermic process. Without management of the exotherm, the temperature may become unacceptably high leading to inoperability of the process and poor yields. The negative impacts of high temperature include rapid catalyst deactivation, low selectivity to ethane and propane, high selectivity to undesirable byproducts such as methane and aromatics, a high rate of coke formation, and metallurgical concerns such as mechanical strength and coking tendency.

In the embodiment as shown in FIG. 1, the process of converting a feed in a series of reactors 101 is described with reference to a NEP process. The NEP process 101 may comprise an NEP reaction section 151 comprising a series of reactors, a heat exchanger 120, a splitter column 170, and a separation unit 180. In an embodiment, the NEP reaction section 151 may comprise a first NEP reactor 130 and a second NEP reactor 160. As shown, a naphtha feed stream in line 102 may be combined with a hydrogen stream in line 111 to provide a charge stream which is charged to the NEP reactor 151. In accordance with the present disclosure, dedicated naphtha streams may be taken from the naphtha feed stream in line 102 for each reactor of the series of reactors. In an exemplary embodiment, a first naphtha stream in line 103, and a second naphtha stream in line 106 may be taken from the naphtha feed stream in line 102. In another aspect, dedicated hydrogen streams may be taken from the hydrogen stream in line 111 for each reactor of the series of reactors. In accordance with another exemplary embodiment, a first hydrogen stream in line 112, and a second hydrogen stream in line 115 may be taken from the hydrogen stream in line 111. The dedicated hydrogen streams may be combined with the dedicated naphtha feed streams to provide a dedicated charge stream for each reactor of the series of reactors.

As shown in FIG. 1, the first naphtha feed stream in line 103 may be combined with a first hydrogen stream in line 112 to provide a dedicated first charge stream in line 108. The first charge stream in line 108 is charged to the dedicated first NEP reactor 130. Similarly, the second naphtha feed stream in line 106 may be combined with a second hydrogen stream in line 115 to provide a dedicated second charge stream in line 118. In an embodiment, each of the first charge stream in line 108, and the second charge stream in line 118 may be heated in the heat exchanger 120 by heat exchange with a reactor effluent stream from one reactor of the series of reactors before charging them to the dedicated reactors. In an aspect, the dedicated charge stream to the dedicated NEP reactor may be heated in the heat exchanger 120 by heat exchange with a reactor effluent stream from the last reactor of the series of reactors. In an exemplary embodiment, each of the first charge stream in line 108, and the second charge stream in line 118 may be the heated in the heat exchanger 120 by heat exchange with a reactor effluent stream from the second NEP reactor 160.

The first charge stream in line 108 is passed to the heat exchanger 120 and heat exchanged with a reactor effluent stream in line 161 from the second NEP reactor 160 to provide a first heated charge stream in line 121. The heat exchanger 120 may comprise a hot inlet side 41 and a hot outlet side 43 for the hot stream, and a cold first inlet side 42 and a cold first outlet side 44 for the cold first charge stream in line 108. Further, the heat exchanger 120 may comprise a cold second inlet side 72 and a cold first outlet side 74 for the cold second charge stream in line 118. A hot reactor effluent stream in line 161 from the second NEP reactor 160 is passed to the hot inlet side 41and after heat exchange is removed from the hot outlet side 43 of the heat exchanger 120. The first charge stream in line 108 is passed to the cold first inlet side 42 and after heat exchange, the first heated charge stream is removed from the cold first outlet side 44 of the heat exchanger 120 in line 121. The first heated charge stream in line 121 is charged to the first NEP reactor 130. The first naphtha feed stream and the first hydrogen stream in the first heated charge stream are contacted with an NEP catalyst in the first NEP reactor 130 to provide a first contacted stream.

In accordance with the present disclosure, the naphtha stream in line 102 may comprise C4 to C12 hydrocarbons preferably having a T10 between about −10° C. and about 60° C. and a T90 between about 70° C. and about 180° C. The naphtha feed stream may comprise normal paraffins, olefins, iso-paraffins, naphthenes, and aromatics. The naphtha stream may be heated to a reaction temperature of about 300° C. to about 600° C., suitably between about 325° C. and about 550° C., and preferably between about 350° C. and about 525° C. Overall weight space velocity, defined herein as hourly mass flow rate of feed in stream 102 divided by total catalyst in all reactors should be between about 0.3 to about 20 hr⁻¹, suitably between about 0.5 and about 10 hr⁻¹ and preferably between about 1 to about 4 hr⁻¹. A total pressure should be about 0.1 to about 3 MPa (abs), or from about 1.5 to about 2.5 MPa (abs), preferably greater than 1 MPa (abs). In an embodiment, the first NEP reactor 130 may be operated at a higher pressure than the other reactors. Under these conditions, C2-C4 yield is consistently in an excess of 80 wt %, while methane yield is less than about 16 wt %, suitably below about 14 wt % and typically below about 12 wt % and preferably no more than 10 wt %.

In accordance with the present disclosure, the first NEP reactor 130, and the second NEP reactor 160 may be operated at similar or different operating conditions.

The hydrogen-to-hydrocarbon molar ratio is important to selectively producing ethane and propane. The hydrogen-to-hydrocarbon ratio should be about 0.3 to about 15 and preferably about 0.5 to about 5. In a further embodiment, the hydrogen-to-hydrocarbon molar ratio may typically be no more than 5, suitably be no more than 3 and preferably be no more than 2. Low hydrogen-to-hydrocarbon ratio promotes desired reaction kinetics which are initiated with dehydrogenation. Hydrogen-to-hydrocarbon ratio may range from about 50% to about 500%, suitably no more than 300% and preferably no more than 200% of stoichiometric requirements to convert naphtha molecules to ethane and/or propane. The molar ratio of hydrogen to hydrocarbon depends on the feed type including paraffin, olefin, naphthene or aromatics, the feed molecular carbon number, and the desired product between predominantly ethane, predominantly propane or ethane and propane of comparable abundance. For example, converting 1 mole of propane to ethane at stoichiometry, the process would require co-feeding 0.5 moles of hydrogen. In practice, the process can operate above or below this stoichiometry of 0.5 such as 0.33 to achieve greater than 40% ethane and less than 15% methane, depending on the process design parameters such as, feed contaminants, reactor type (fixed bed, moving bed, fluidized bed), and regeneration frequency. As the carbon number of feed molecules increases from light naphtha (C₄-C₇) to full range naphtha (C₆-C₁₀) the amount of hydrogen required for the reaction increases.

The NEP catalyst for converting naphtha to ethane and propane may contain a molecular sieve comprising large or medium pore mouths, that is, comprising 10 or 12 member rings, respectively. Examples of suitable molecular sieves include MFI, MEL, MFI/MEL intergrowth, MTW, TUN, UZM-39, IMF, UZM-44, UZM-54, MWW, UZM-37, UZM-8, UZM-8HS. Examples of suitable molecular sieves further include FER, AHT, AEL (SAPO-11), AFO (SAPO-41), MRE, MFS, EUO-1, TON (ZSM-22), MTT (ZSM-23) and UZM-53. Additional molecular sieves with larger pores include FAU, EMT, FAU/EMT intergrowth, UZM-14, MOR, BEA, UZM-50, MTW, ZSM-12. Additional examples include MSE and UZM-35.

MFI is a suitable NEP catalyst. It will be appreciated that ZSM-5 is an MFI-type aluminosilicate zeolite belonging to the pentasil family of zeolites and having a chemical formula of NaₙA1ₙSi₉₆-nO₁₉₂·16H₂O (0<n<10). In various embodiments, the ZSM-5 zeolite may comprise a silica-to-alumina molar ratio of 20 to 1000, 20 to 800, 20 to 600, 20 to 400, 20 to 200 or 20 to 80. In various embodiments, the ZSM-5 zeolite may comprise a crystal size in the range of 10 to 600 nm, 20 to 500 nm, 30 to 450, 40 to 400 nm, or 50 to 300 nm.

The NEP catalyst may comprise a bound zeolite. The binder may comprise an oxide of aluminum, silicon, zinc, titanium, zirconium and mixtures thereof. The binder may comprise a phosphate in the binder or a phosphate of the forenamed oxide binder materials. Preferably, the binder is a silicon oxide. The MFI zeolite may be supported in a silicon oxide containing binder or an alumina containing binder such as aluminum phosphate.

MFI zeolite slurry may be first mixed with a binder in the form of colloidal suspension (sol) and gelling reagent and then dropped into hot oil to make spheres controlled to produce ⅛-inch to about 1/32-inch diameter calcined supports. Alternatively, the zeolite may be mixed with a silicon oxide containing binder and extruded to 1/32 to ¼ inch diameter extrudates. Extrudates may be washed with ammonia to remove sodium ions from the zeolite, dried and calcined to remove the organic structural directing agent (OSDA) from the synthesized zeolite. Optionally, the calcined support may be ammonium-ion exchanged using an ammonium nitrate solution to remove residual sodium ions and dried at about 110° C.

The NEP catalyst comprises a metal on the catalyst. The metal may comprise a transition metal. In a further example, the metal may comprise platinum, palladium, iridium, rhenium, ruthenium and mixtures thereof. The metal may be a noble metal. A modifier metal may also be included on the catalyst. The modifier metal may include tin, germanium, gallium, indium, thallium, zinc, silver and mixtures thereof. The modifier metal should be more concentrated on the binder than on the zeolite. About 0.01 to about 5 wt % of each of the transition metal and the modifier metal may be on the catalyst.

Metal may be incorporated into the binder by evaporative impregnation. A solution of platinum such as tetraamine platinate nitrate or chloroplatinic acid may be contacted with the bound spherical or extrudate supports which have been calcined and ion-exchanged in a rotary evaporator, followed by drying and oxidation.

The NEP catalyst comprises a metal on the bound spherical or extrudate supports of the catalyst. Preferably, more of the metal is on the binder than on the zeolite. At least 60 wt %, suitably at least 70 wt %, preferably at least 80 wt % and most preferably at least 90 wt % of the metal is on the binder. The zeolite and/or the entire NEP catalyst may be steamed oxidized to drive the metal off the zeolite. Steaming is preferably effected after the metal is added to the catalyst. The dried, impregnated spherical or extrudate supports may be steam oxidized in air for sufficient time to provide NEP catalysts. Steam oxidation in air at a temperature of about 500° C. to about 650° C. and about 5 mol % to about 30 mol % steam for about 1 to 3 hours may be suitable.

The NEP catalysts must be reduced to activate them for catalyzing the NEP reaction. For example, the catalyst may be reduced in flowing hydrogen at about 500 to about 550° C. for 3 hours before contacting feed.

In accordance with the present disclosure, the first NEP reactor 130, and the second NEP reactor 160 may comprise similar or different NEP catalysts.

After conversion, a first reactor effluent stream is discharged from the first NEP reactor 130 in line 131. The first reactor effluent stream in line 131 may be a first product stream. The first reactor effluent stream in line 131 may be passed to a heat exchanger 10 to cool the stream, perhaps generating steam. A cooled first reactor effluent stream is taken in line 132 from the heat exchanger 10 and charged to the next downstream reactor of the series of reactors. Alternatively, the heat removal between the stages may include producing superheated steam or heating of another process stream within or outside the current process.

The second charge stream in line 118 is passed to the heat exchanger 120 and heat exchanged with the reactor effluent stream in line 161 from the second NEP reactor 160 to provide a second heated charge stream in line 128. The hot reactor effluent stream in line 161 from the second NEP reactor 160 is passed to the hot inlet side 41 and after heat exchange is removed from the hot outlet side 43 of the heat exchanger 120. The second charge stream in line 118 is passed to a cold second inlet side 72 and after heat exchange, a second heated charge stream is removed from a cold second outlet side 74 of the heat exchanger 120 in line 128. The second heated charge stream in line 128 is charged to the second NEP reactor 160. The second naphtha feed stream and the second hydrogen stream in the second heated charge stream are contacted with an NEP catalyst in the second NEP reactor 130 to provide a second contacted stream.

After conversion, the second reactor effluent stream is taken in line 161 from the second NEP reactor 160. The second reactor effluent stream in line 161 has significant amount of heat which can be recovered. The second reactor effluent stream in line 161 may be passed to the heat exchanger 120 to heat the dedicated naphtha stream for each reactor of the series of reactors. The second reactor effluent stream in line 161 may be the last contacted stream of the series of NEP reactors of the reaction section 151.

In an embodiment, the series of reactors in the reaction section 151 may further comprise a third NEP reactor 140 and a fourth NEP reactor 150 in series with the first NEP reactor 130 and second NEP reactor 140. In the exemplary embodiment as shown in FIG. 1, the third NEP reactor 140 and the fourth NEP reactor 150 may be in downstream fluid communication with the first NEP reactor 130 and in upstream fluid communication with the second NEP reactor 140.

A third naphtha feed stream in line 104 for the third NEP reactor 140 may be taken from the naphtha feed stream in line 102. A third hydrogen stream in line 113 for the third NEP reactor 140 may be taken from the hydrogen stream in line 111. As shown in FIG. 1, the third naphtha feed stream in line 104 may be combined with the third hydrogen stream in line 113 to provide a dedicated third charge stream in line 109. The third charge stream in line 109 is charged to the dedicated third NEP reactor 140.

The third charge stream in line 109 is passed to the heat exchanger 120 and heat exchanged with a hot second reactor effluent stream in line 161 from the second NEP reactor 160 to provide a third heated charge stream in line 126. The heat exchanger 120 may comprise a cold third inlet side 82 and a cold third outlet side 84 for the cold third charge stream in line 109. The hot second reactor effluent stream in line 161 from the second NEP reactor 160 is passed to the hot inlet side 41 and after heat exchange is removed from the hot outlet side 43 of the heat exchanger 120. The third charge stream in line 109 is passed to the cold third inlet side 82 and after heat exchange, the third heated charge stream is removed from the cold third outlet side 84 of the heat exchanger 120 in line 126.

The third heated charge stream in line 126 is charged to the third NEP reactor 140. The third naphtha feed stream and the third hydrogen stream in the third heated charge stream are contacted with an NEP catalyst in the third NEP reactor 140 to provide a third reactor effluent stream.

In an exemplary embodiment, the third heated charge stream in line 126 may be combined with the cooled first reactor effluent stream in line 132 to provide a combined third charge stream in line 133. The combined third charge stream in line 133 is charged to the third NEP reactor 140.

In an alternate embodiment, the third charge stream in line 109 may be combined with the cooled first reactor effluent stream in line 132 to provide a combined third charge stream. The combined third charge stream may be heated by heat exchange with the hot second reactor effluent stream in line 161 in the heat exchanger 120 provide a heated combined third charge stream. The heated combined third charge stream may be charged to the third NEP reactor 140.

In the third NEP reactor 140, the cooled first reactor effluent stream and the third charge stream are contacted with an NEP catalyst to provide a third reactor effluent stream in line 142. The third reactor effluent stream in line 142 may be a third product stream. The NEP catalyst in the third NEP reactor 140 can be similar or a different NEP catalyst than the first NEP reactor 130. In accordance with the present disclosure, the third NEP reactor 140 may be operated at similar or different operating conditions than the first NEP reactor 130 and the second NEP reactor 160.

After conversion, a third reactor effluent stream is taken in line 142 from the third NEP reactor 140. The third reactor effluent stream in line 142 may be passed to a heat exchanger 20 to generate steam. A heat exchanged perhaps cooled third reactor effluent stream is taken in line 143 from the heat exchanger 20. The cooled third reactor effluent stream in line 143 may be charged to the fourth NEP reactor 150. Alternatively, the heat removal between the stages may be via superheating of steam or heating of another process stream within or outside the current process.

A fourth naphtha feed stream in line 105 for the fourth NEP reactor 150 may be taken from the naphtha feed stream in line 102. A fourth hydrogen stream in line 114 for the fourth NEP reactor 150 may be taken from the hydrogen stream in line 111. As shown in FIG. 1, the fourth naphtha feed stream in line 105 may be combined with the fourth hydrogen stream in line 114 to provide a dedicated fourth charge stream in line 116. The fourth charge stream in line 116 is charged to the dedicated fourth NEP reactor 150.

The fourth charge stream in line 116 is also passed to the fourth NEP reactor 150. The fourth charge stream in line 116 may be heated in the heat exchanger 120 to provide a heat exchanged perhaps heated fourth charge stream which is passed to the fourth NEP reactor 150. The fourth charge stream in line 116 is passed to the heat exchanger 120 and heat exchanged with the hot second reactor effluent stream in line 161 from the second NEP reactor 160 to provide a fourth heated charge stream in line 127. The heat exchanger 120 may comprise a cold fourth inlet side 62 and a cold fourth outlet side 64 for the cold fourth charge stream in line 116. The hot second reactor effluent stream in line 161 from the second NEP reactor 160 is passed to the hot inlet side 41 and after heat exchange is removed from the hot outlet side 43 of the heat exchanger 120. The fourth charge stream in line 116 is passed to the cold fourth inlet side 62 and after heat exchange, the fourth heated charge stream is removed from the hot fourth outlet side 64 of the heat exchanger 120 in line 127.

The fourth heated charge stream in line 127 is charged to the fourth NEP reactor 150. The fourth naphtha feed stream and the fourth hydrogen stream in the fourth heated charge stream are contacted with an NEP catalyst in the fourth NEP reactor 150 to provide a fourth reactor effluent stream.

In an exemplary embodiment, the fourth heated charge stream in line 127 may be combined with the cooled third reactor effluent stream in line 143 to provide a combined fourth charge stream in line 144. The combined fourth charge stream in line 144 is charged to the fourth NEP reactor 150.

In an alternate embodiment, the fourth charge stream in line 116 may be combined with the cooled third reactor effluent stream in line 143 to provide a combined fourth charge stream. The combined fourth charge stream may be heated by heat exchange with the hot second reactor effluent stream in line 161 in the heat exchanger 120 provide a heated combined fourth charge stream. The heated combined fourth charge stream may be charged to the fourth NEP reactor 150.

In the fourth NEP reactor 150, the cooled third contacted stream and the fourth charge stream are contacted with an NEP catalyst to provide a fourth reactor effluent stream in line 152. The fourth reactor effluent stream in line 142 may be a fourth product stream. The NEP catalyst in the fourth NEP reactor 150 can be similar or a different NEP catalyst than the first NEP reactor 130 and the third NEP reactor 150.

After conversion, a fourth reactor effluent stream is taken in line 152 from the fourth NEP reactor 150. The fourth reactor effluent stream in line 152 may be passed to a heat exchanger 30 to generate steam. A heat exchanged perhaps cooled fourth reactor effluent stream is taken in line 153 from the heat exchanger 30. Alternatively, the heat removal between the stages may be via superheating of steam or heating of another process stream within or outside the current process. The cooled fourth reactor effluent in line 153 may be charged to the last reactor of the series of reactors. Alternatively, the cooled fourth reactor effluent stream in line 153 may be combined with a dedicated last charge stream of the series of reactors and charged to the last reactor.

In exemplary embodiment as shown in FIG. 1, the second reactor 160 may be the last reactor of the series of reactors. The second naphtha feed stream in line 106 for the second NEP reactor 160 may be taken from the naphtha feed stream in line 102. As shown in FIG. 1, the second naphtha feed stream in line 106 may be combined with the second hydrogen stream in line 115 to provide a dedicated second charge stream in line 118. The second charge stream in line 118 is charged to the dedicated second NEP reactor 160.

The second charge stream in line 118 is passed to the heat exchanger 120 and heat exchanged with the hot second reactor effluent stream in line 161 from the second NEP reactor 160 to provide a second heated charge stream in line 128. The heat exchanger 120 may comprise a cold second inlet side 72 and a hot second outlet side 74 for the cold second charge stream in line 118. The hot second reactor effluent stream in line 161 from the second NEP reactor 160 is passed to the hot inlet side 41 and after heat exchange is removed from the hot outlet side 43 of the heat exchanger 120. The second charge stream in line 118 is passed to the cold second inlet side 72 and after heat exchange, the second heated charge stream is removed from the cold second outlet side 74 of the heat exchanger 120 in line 128.

The second heated charge stream in line 128 is charged to the second NEP reactor 160. The second naphtha feed stream and the second hydrogen stream in the second heated charge stream are contacted with an NEP catalyst in the second NEP reactor 160 to provide the second contacted stream. The NEP catalyst in the second NEP reactor 10 can be similar or a different NEP catalyst than the first NEP reactor 130, the third NEP reactor 140, and the fourth NEP reactor 150.

In an exemplary embodiment, the second heated charge stream in line 128 may be combined with the cooled fourth reactor effluent stream in line 153 to provide a combined second charge stream in line 154. The combined second charge stream in line 154 is charged to the second NEP reactor 160.

In an alternate embodiment, the second charge stream in line 118 may be combined with the cooled fourth reactor effluent stream in line 153 to provide a combined second charge stream. The combined second charge stream may be heated by heat exchange with the hot second reactor effluent stream in line 161 in the heat exchanger 120 provide a heated combined second charge stream. The heated combined second charge stream may be charged to the second NEP reactor 160.

In the second NEP reactor 140, the cooled fourth reactor effluent stream and the second heated charge stream are contacted with an NEP catalyst to provide the third reactor effluent stream in line 161. The second reactor effluent stream in line 161 may be a third product stream. In an aspect, the second reactor effluent stream in line 161 may be a final product stream from the series of reactors. The NEP catalyst in the second NEP reactor 160 can be similar or a different NEP catalyst than the first NEP reactor 130, the third NEP reactor 140, and the fourth NEP reactor 150. The second NEP reactor 160 may be operated at similar or different operating conditions than the first NEP reactor 130, the third NEP reactor 140 and the fourth NEP reactor 150.

In accordance with the present disclosure, the second reactor effluent stream in line 161 may be a last stage or reactor contacted stream of the series of reactors.

The present disclosure includes preheating the dedicated naphtha streams before charging them to their dedicated NEP reactors. The first naphtha stream in line 103, the second naphtha stream in line 106, the third naphtha stream in line 104, and the fourth naphtha stream in line 105 are all heat exchanged with one of the NEP reactor effluent streams. In accordance with the present disclosure, the first naphtha stream in line 103, the second naphtha stream in line 106, the third naphtha stream in line 104, and the fourth naphtha stream in line 105 are heat exchanged with a reactor effluent stream from the last NEP reactor of the series of NEP reactors.

The chemistry of the NEP process is exothermic and requires heat management of the released heat. Typical approach of heat management includes adding dedicated feed or reactor effluent exchangers for each reactor of the series of reactors. This may increase the total equipment and cost. Also, it may lead to a more difficult process control scheme because the of reactor effluent for heat exchange would need to be accurately split for dedicated feed or reactor effluent exchangers for each reactor to control temperature rise in each reactor. Temperature control with the split of reactor effluent stream cannot be independent since increasing flow rate in one split may decrease flow of effluent in another split, which may affect the temperature control in the split path with decreased flow. Complicated process control affects yield such as byproduct methane would increase in NEP process with poor control as well as process safety as temperature excursions can lead to reactor light-off in a positive feedback loop.

The present process address this problem and provides a single heat exchanger 120 for heating the dedicated reactor feed streams with the reactor effluent stream from the last reactor of the series of the reactors. This approach would reduce the equipment cost and makes the temperature control more efficient and easier to implement. Also, it would reduce the yield of sometimes undesirable components. Though, this approach is shown in the exemplary embodiment of FIG. 1 for an NEP process, the approach is equally applicable for other exothermic processes with series of reactors. Such other processes may include a methanol to jet process, and an ethanol to jet process. In an aspect of the present disclosure, the heat exchanger 120 may be a spiral tube heat exchanger. In another aspect of the present disclosure, the heat exchanger 120 may be a plate heat exchanger which includes plate and frame heat exchangers and welded plate heat exchangers.

A plate heat exchanger can include many types of plate heat exchangers. In some case, a frame may not be used while the exchanger is still considered to be within the category of plate heat exchangers. In one example, the plates may be sealed from each other and externally by gaskets and the gaskets may be seated by a frame. In another example the plates can be sealed by welding, providing sealing both between the fluids and externally, and a frame may or may not be used in the design. Plates are gasketed together or welded together in a bundle which make up channels that allow separate fluid flow, enables heat transfer between adjacent channels carrying a different fluids.

Headers may be attached to the plate bundles that allow streams to flow in specific plate channels in the heat exchanger. For example, there may be an inlet manifold and an outlet manifold to distribute flow to channels between plates for a hot fluid. There may also be an inlet manifold and an outlet manifold to distribute flow to channels between plates for a first cold fluid, and still further inlet and outlet manifolds for a second, and additional cold fluids. The arrangement of these plates and manifolds allow these hot and cold streams to exchange heat with each other, while keeping the fluids separate from inlet through outlet.

Referring back to FIG. 1, the second reactor effluent stream in line 161 may be a light paraffin stream. The light paraffin stream in line 161 may comprise at least about 40 wt % ethane or at least about 40 wt % propane or at least about 70 wt % and preferably at least about 80 wt % ethane and propane. The ethane to propane ratio can range from about 0.1 to about 5. The light paraffin stream can have less than about 16 wt %, suitably less than about 14 wt %, preferably less than about 12 wt %, and more preferably less than about 10 wt % methane.

After heat exchange with the dedicated charge streams in the heat exchanger 120, a cooled light paraffin stream may be discharged in line 162 from the hot outlet side 43 of the heat exchanger 120. In an embodiment, the cooled light paraffin stream in line 162 may be fed to the splitter column 170. In an exemplary embodiment, the splitter column 170 is a fractionation column that separates ethane and propane from aromatics. In the splitter column 170, the ethane and propane along with other light gases may be separated in an overhead stream in line 174. The aromatics are separated into a bottoms stream in line 175. The bottoms stream in line 175 may comprise single or multi-ring aromatics.

The overhead stream in line 174 is passed to an NEP separation unit 180 to separate ethane and propane. The NEP separation unit 180 may be a fractionation column or a series of fractionation columns and other separation units that may separate the overhead stream in line 174 into the hydrogen stream in line 181, an ethane stream in line 182, a propane stream in line 183 and the heavy stream in line 184. The NEP separation unit 180 may comprise a compressor to increase pressure suitable for downstream separation. The NEP separation unit 180 may comprise a demethanizer column that separates the overhead light paraffin stream into a gas stream in an overhead line and a C2+ paraffin stream in a bottoms line. The gas stream may be sent to a hydrogen purification unit such as a PSA unit to recover hydrogen in line 181 for recycle to the NEP reactor 151. Remaining methane from the hydrogen purification unit may be used for fuel gas. The C2+ paraffin stream may then be fed to a deethanizer column to produce the ethane stream in a deethanizer overhead line 182 and a C3+ paraffin stream in a deethanized bottoms line. The C3+ paraffin stream may then be fed to a depropanizer column to produce the propane stream in a depropanizer overhead line 183 and the heavy paraffin stream in the recycle line 184 which may comprise C4+ hydrocarbons. The NEP separation unit 180 may take other forms.

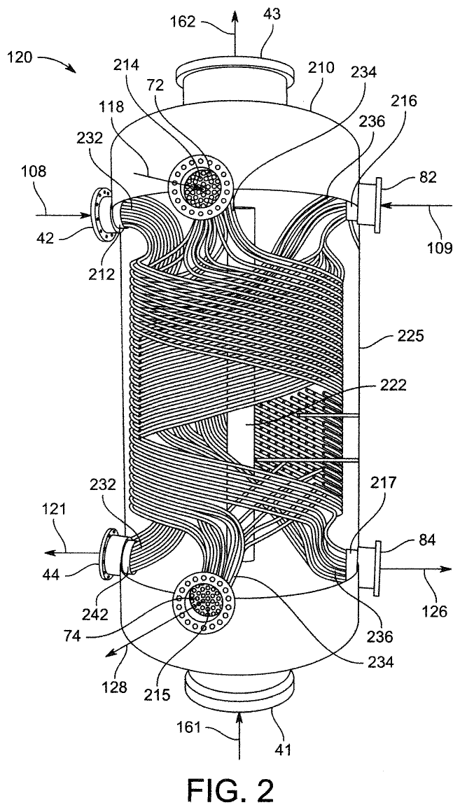

FIG. 2 illustrates an exemplary embodiment of the heat exchanger 120 from FIG. 1. In the exemplary embodiment as shown in FIG. 2, the heat exchanger 120 comprises a spiral tube heat exchanger. Spiral tube heat exchanger is a less common type of heat exchanger. This heat exchanger is composed of heat exchange tubes coiled in a spiral pattern. Layers of spiral wound tube, which may also be called coil wound tubes, are wrapped around a small central core or mandrel. As more tubes are added to the heat exchanger, additional wraps of the spiral wound tubes are added. The tubes may be connected to one or more inlet tubesheets and one or more outlet tubesheets, to allow one or more fluids to flow on the tube side of the heat exchanger. The tube side flow or flows would then exchange heat with a separate flow of fluid on the outside of the tubes, also called the shell side fluid. The tube side fluid or fluids and the shell side fluid may be arranged in either a co-current flow arrangement, or more preferably in this application in a counter-current flow arrangement.

The spiral tube heat exchanger has many advantages over the typical heat exchangers types.

The curvature of the spiral tubes creates a secondary flow between the inner and outer portions of the flow path and ensures highly turbulent fluid flow throughout the tubes. The secondary flow increases mixing which also enhances heat transfer. Due to the arrangement of the spiral tubes, the temperature distribution is even throughout the device, and there are no hot or cold spots, and no low flow or stagnant flow regions, as with typical shell and tube type heat exchangers.

The constantly changing curvature of the spiral tube heat exchanger also ensures highly turbulent fluid flow throughout the path. This increases mixing which also enhances heat transfer and the temperature distribution is even throughout the device. There are no hot and cold spots as with other heat exchanger types which further improves the exchanger's performance. The long inside flow length of the spiral tubes in counter-current flow arrangement with the shellside flow provides better velocities and heat transfer coefficients, as well as better use of pressure drops than can be achieved with conventional straight-tube shell and tube type heat exchangers.

FIG. 2 provides an embodiment of heat exchanger 120 as a spiral tube heat exchanger. In the following description, the working of the heat exchanger 120 is provided using a spiral tube heat exchanger in reference to the NEP process from FIG. 1. However, the spiral tube heat exchanger of FIG. 2 is equally applicable with other exothermic processes such as a methanol to jet fuel process and an ethanol to jet fuel process. Likewise, a plate exchanger is equally applicable as an embodiment of heat exchanger 120 and can provide the fluid communication of the multiple streams and the heat transfer as described here with spiral tube heat exchangers.

Referring to FIG. 2, the spiral tube heat exchanger 120 is in fluid communication with the NEP reaction section 151, particularly the series of reactors in the NEP reaction section 151. In an aspect, the spiral tube heat exchanger 120 is in fluid communication with all the four NEP reactors, the first NEP reactor 130, the second NEP reactor 160, the third NEP reactor 140, and the fourth NEP reactor 150. For simplicity, the working of the spiral tube heat exchanger 120 is provided with reference to the three NEP reactors from FIG. 1, the first NEP reactor 130, the second NEP reactor 160, and the third NEP reactor 140.

In an aspect, the spiral tube heat exchanger 120 comprises at least two tube sheets. In the embodiment as shown in FIG. 2, the spiral tube heat exchanger 120 may comprise four tube sheets. For the reference purpose, three tube sheets are shown, a first tube sheet 212, a second tube sheet 214, and a third tube sheet 216. Each of the tube sheets may be in fluid communication with a plurality of tubes. As shown, the first tube sheet 212 may be in fluid communication with a first plurality of tubes 232, the second tube sheet 214 may be in fluid communication with a second plurality of tubes 234, and the third tube sheet 216 may be in fluid communication with a third plurality of tubes 236. Each of the first plurality of tubes 232, the second plurality of tubes 234, and the third plurality of tubes 236 may comprise one or more tube sheets. In an exemplary embodiment, each of the first plurality of tubes 232, the second plurality of tubes 234, and the third plurality of tubes 236 may comprise at least two tube sheets. The first plurality of tubes 232 may be in communication with a first inlet tube sheet 212 and a first outlet tube sheet 242. The second plurality of tubes 234 may be in communication with a second inlet tube sheet 214 and a second outlet tube sheet 215. The third plurality of tubes 236 may be in communication with a third inlet tube sheet 216 and a third outlet tube sheet 217.

The spiral tube heat exchanger 120 may comprise the plurality of tubes, the first plurality of tubes 232, the second plurality of tubes 234, and the third plurality of tubes 236 wound helically over a mandrel or core 222 and placed inside a shell 210 as shown in FIG. 2. In an aspect, the spacing of tubes in each of the first plurality of tubes 232, the second plurality of tubes 234, and the third plurality of tubes 236 may be uniform. In an embodiments, spacers may be provided between the adjacent tubes in the plurality of tubes. In a further aspect, the length of each of the tubes in the first plurality of tubes 232, the second plurality of tubes 234, and the third plurality of tubes 236 may be similar. However, the length of the tubes may be selected as per suitability.

The hot reactor effluent stream form the last reactor, which is the second reactor effluent stream in line 161 from the second NEP reactor 160 may be passed to the shell side of the spiral tube heat exchanger 120 through the hot inlet side 41.

The first charge stream in line 108 is passed to the spiral tube heat exchanger 120 through the cold first inlet side 42. The first charge stream in line 108 flows through the first inlet tube sheet 212 to the first plurality of tubes 232. The first charge stream flowing in the first plurality of tubes 232 is heated after heat exchange with the second reactor effluent stream in line 161 from the second NEP reactor 160. The second reactor effluent stream in line 161 flows in the shell side 225 of the spiral tube heat exchanger 120. The hot second reactor effluent stream in line 161 may be in direct contact with an outer side of the tubes. The indirect heat exchange from the hot second reactor effluent stream in line 161 to the first charge stream flowing in the first plurality of tubes 232 heats up the first charge stream while it flows from the inlet to the outlet side. The first charge stream in line 108 flows from the first plurality of tubes 232 through first outlet tube sheet 242. A first heated charge stream may exit the first outlet tube sheet 242 and discharged in line 121 from the hot first outlet side 44 of the spiral tube heat exchanger 120. The first heated charge stream is charged to the first NEP reactor 120 and processed as described in FIG. 1.

The second charge stream in line 118 is passed to the spiral tube heat exchanger 120 through the cold second inlet side 72. The second charge stream in line 118 flows through the second inlet tube sheet 214 to the second plurality of tubes 234. The second charge stream flowing in the second plurality of tubes 234 is heated by heat exchange with the second reactor effluent stream in line 161 from the second NEP reactor 160. The second reactor effluent stream in line 161 flows in the shell side 225 of the spiral tube heat exchanger 120. The hot second reactor effluent stream in line 161 may be in direct contact with an outer side of the tubes. The indirect heat exchange from the hot second reactor effluent stream in line 161 to the second charge stream flowing in the second plurality of tubes 234 heats up the second charge stream while it flows from the inlet to the outlet side. The second charge stream in line 118 flows from the second plurality of tubes 234 through second outlet tube sheet 215. A second heated charge stream may exit the second outlet tube sheet 215 and discharged in line 128 from the hot second outlet side 74 of the spiral tube heat exchanger 120. The second heated charge stream in line 128 is charged to the second NEP reactor 160 and processed as described in FIG. 1.

The third charge stream in line 109 is passed to the spiral tube heat exchanger 120 through the cold third inlet side 82. The third charge stream in line 109 flows through the third inlet tube sheet 216 to the third plurality of tubes 236. The third charge stream flowing in the third plurality of tubes 236 is heated after heat exchange with the second reactor effluent stream in line 161 from the second NEP reactor 160. The second reactor effluent stream in line 161 flows in the shell side 225 of the spiral tube heat exchanger 120. The hot second reactor effluent stream in line 161 may be in direct contact with an outer side of the tubes. The indirect heat exchange from the hot second reactor effluent stream in line 161 to the third charge stream flowing in the third plurality of tubes 236 heats up the third charge stream while it flows from the inlet to the outlet side. The third charge stream in line 109 flows from the third plurality of tubes 236 through third outlet tube sheet 217. A third heated charge stream may exit the third outlet tube sheet 217 and discharged in line 126 from the hot third outlet side 84 of the spiral tube heat exchanger 120. The third heated charge stream in line 126 is charged to the third NEP reactor 180 and processed as described in FIG. 1.

Although not shown in FIG. 2, the spiral tube heat exchanger may comprise a fourth inlet tube sheet and a fourth outlet tube sheet in communication with a fourth plurality of tubes. The fourth charge stream in line 116 may be passed to the fourth inlet tube sheet and heated in the fourth plurality of tubes with the hot second reactor effluent stream in line 161. The

[symbol]

The fourth charge stream in line 116 may be heated similarly in the spiral tube heat exchanger 120 to provide the fourth heated charge stream in line 127 and charged to the fourth NEP reactor 150 as described earlier in FIG. 1.

In the spiral tube heat exchanger 120, the hot second reactor effluent stream in line 161 flows across the wound plurality tubes in the space between the mandrel 222 and an inner side of the shell 110 which is defined as the shell side 225. The counter-counter flow arrangement of the fluids in the spiral tube heat exchanger 120 provides an efficient heat exchange from the hot fluid to the colder fluids. The coiling of the tubes as shown in FIG. 2, ensures a large cross-sectional area and a small length of flow on the low-pressure shell side 225 and a large length of tubes with small cross-sectional area on the high-pressure side which is the tube side. This way, a high heat transfer coefficient can be maintained on the tube side with efficacy.

The heat exchanger 120, as a plate heat exchanger, or as a spiral tube heat exchanger as shown in FIG. 2, can be easily applied to any exothermic process. The heat exchanger 120 reduces equipment count and cost by combining multiple exchangers into a single exchanger. This also improves process control and safety because there is no need to accurately split the hot reactor effluent into different streams for heating multiple feeds in a series of reactors. Instead, process control is achieved by varying heat duty in the feed to achieve the appropriate feed inlet temperature and finer temperature control. This control is much simpler and safer because it replaces a highly dependent control scheme of splitting reactor effluent with an orthogonalized, independent control scheme. These same benefits are realized for replacing the dedicated heat exchangers that heat separate feeds for separate charge heaters and reactors by a single plate exchanger or single spiral-wound exchanger in the ethanol to jet (ETJ) process and the methanol to jet (MTJ) process. This configuration would provide reduced equipment count, cheaper equipment cost, and reduced plot space.

### EXAMPLES

### Example 1

The present process with a single heat exchanger for heating the four feed streams was compared with the conventional process. The conventional process required four shell and tube heat exchangers to heat four separate feed streams. In the conventional process, a feed stream to a first reactor was about 85,300 kg/hr. A first feed stream was heated by about 103,700 kg/hr of reactor effluent, transferring about 23.6 MW of heat from a first effluent stream to the first feed stream. The first heat exchanger was a cylindrical vessel, with the first feed stream flowing through the tube side, and an effluent stream flowing through the shell side. The first heat exchanger was about 23.3 meters long and about 1.25 meters in diameter. A second feed stream to a second reactor was about 104,000 kg/hr. The second feed stream was heated by about 124,500 kg/hr of reactor effluent, transferring about 28.3 MW of heat from a second effluent stream to the second feed stream. The second heat exchanger was a cylindrical vessel, with the second feed stream flowing through the tube side, and the effluent stream flowing through the shell side. The second heat exchanger was about 23.4 meters long and about 1.27 meters in diameter. A third feed stream to a third reactor was about 134,900 kg/hr. The third feed stream was heated by about 159,900 kg/hr of reactor effluent, transferring about 36.3 MW of heat from a third effluent stream to the third feed stream. The third heat exchanger was a cylindrical vessel, with the third feed stream flowing through the tube side, and the effluent stream flowing through the shell side. The third heat exchanger was about 23.5 meters long and about 1.43 meters in diameter. A fourth feed stream to a fourth reactor was about 196,800 kg/hr. The fourth feed stream was heated by about 230,600 kg/hr of reactor effluent, transferring about 52.4 MW of heat from a fourth effluent stream to the fourth feed stream. The fourth heat exchanger was a cylindrical vessel, with the fourth feed stream flowing through the tube side, and the effluent stream flowing through the shell side. The fourth heat exchanger was about 23.7 meters long and about 1.73 meters in diameter. Because the fraction of the total effluent flow to each shell and tube heat exchanger is different, it could not be split by symmetrical piping alone, and the effluent stream required control valves to provide the correct flow to each heat exchanger.

In the present process, the four feed streams were heated in a single welded plate bundle of a plate heat exchanger. The four feed streams were entered from four separate manifolds on a single welded plate bundle and exited four separate manifolds at the opposite end of that single welded plate bundle. The total reactor effluent flow stream of about 618.7 kg/hr entered a manifold at one end of the welded plate bundle and exited through a manifold at the other end of the welded plate bundle and transferred a total of about 140.6 MW of heat to the four separate feed streams. The amount of heat transferred to each feed stream can be set by the design of the plate bundle, specifically by providing the necessary amount of surface area required for each feed stream. This welded plate exchanger was provided in a single cylindrical vessel of about 15.7 meters long and 2.70 meters in diameter.

### Example 2

The four feed streams as described herein above in Example 1, were heated in a single spiral tube heat exchanger. The four feed streams entered the four separate tubesheets on a single spiral tube heat exchanger and exited four separate tubesheets at the opposite end of that single spiral tube heat exchanger. The total reactor effluent flow rate of about 618.7 kg/hr entered the shell side through a nozzle at one end of the spiral tube heat exchanger and exited the shell side through another nozzle at the other end of the spiral tube heat exchanger, and transferred a total of about 140.6 MW of heat to the four separate feed streams inside the tubes. The amount of heat transferred to each feed stream can be set by the design of the spiral tube bundle, specifically by providing the necessary amount of surface area required for each stream. This spiral tube heat exchanger was provided in a cylindrical vessel of about 17.5 meters long and 3.40 meters in diameter.

### Specific Embodiments

While the following is described in conjunction with specific embodiments, it will be understood that this description is intended to illustrate and not limit the scope of the preceding description and the appended claims.

A first embodiment of the present disclosure is a process for converting a feed in a series of reactors, comprising taking at least two hydrocarbon streams, a first hydrocarbon feed stream and a second hydrocarbon feed stream; heat exchanging the first hydrocarbon feed stream and the second hydrocarbon feed stream in a heat exchanger with a second product stream from a second reactor; and charging a heat exchanged first hydrocarbon feed stream into a first catalyst bed in a first reactor to produce a first product stream and a heat exchanged second hydrocarbon feed stream into a second catalyst bed in the second reactor to produce the second product stream. An embodiment of the present disclosure is one, any or all of prior embodiments in this paragraph up through the first embodiment in this paragraph, wherein the heat exchanger is a spiral tube heat exchanger comprising at least two inlet tube sheets surrounded by a shell. An embodiment of the present disclosure is one, any or all of prior embodiments in this paragraph up through the first embodiment in this paragraph further comprising passing the first hydrocarbon feed stream to a first tube sheet and passing the second hydrocarbon feed stream to a second tube sheet; and passing the second product stream to a shell side of the heat exchanger to heat exchange with the first hydrocarbon feed stream and the second hydrocarbon feed stream to produce a heat exchanged second product stream. An embodiment of the present disclosure is one, any or all of prior embodiments in this paragraph up through the first embodiment in this paragraph, wherein the heat exchanger is plate heat exchanger. An embodiment of the present disclosure is one, any or all of prior embodiments in this paragraph up through the first embodiment in this paragraph further comprising a third reactor and comprising combining the first product stream from the first reactor with a third hydrocarbon feed stream to produce a combined third hydrocarbon feed stream; heat exchanging the combined third hydrocarbon feed stream or the third hydrocarbon feed stream in the heat exchanger by heat exchanging with the second product stream from the second reactor; and charging a heat exchanged combined third hydrocarbon feed stream to a third reactor to produce a third product stream. An embodiment of the present disclosure is one, any or all of prior embodiments in this paragraph up through the first embodiment in this paragraph further comprising a fourth reactor and comprising combining the third product stream from the third reactor with a fourth hydrocarbon feed stream to produce a combined fourth hydrocarbon feed stream; heat exchanging the combined fourth hydrocarbon feed stream or the fourth hydrocarbon feed stream in the heat exchanger by heat exchanging with the second product stream from the second reactor; and charging a heat exchanged combined fourth hydrocarbon feed stream to a fourth reactor to produce the fourth product stream An embodiment of the present disclosure is one, any or all of prior embodiments in this paragraph up through the first embodiment in this paragraph further comprising combining the fourth product stream from the fourth reactor with the second hydrocarbon feed stream to produce a combined second hydrocarbon feed stream; heat exchanging the combined second hydrocarbon feed stream or the second hydrocarbon feed stream in the heat exchanger by heat exchanging with the second product stream from the second reactor; and charging a heat exchanged combined second hydrocarbon feed stream to the second reactor to produce the second product stream. An embodiment of the present disclosure is one, any or all of prior embodiments in this paragraph up through the first embodiment in this paragraph, wherein the second product stream is at a higher temperature than the first hydrocarbon feed stream and the second hydrocarbon feed stream. An embodiment of the present disclosure is one, any or all of prior embodiments in this paragraph up through the first embodiment in this paragraph, wherein the process comprises converting a naphtha stream to produce ethane and propane in the series of reactors. An embodiment of the present disclosure is one, any or all of prior embodiments in this paragraph up through the first embodiment in this paragraph, wherein the process comprises oligomerizing the hydrocarbon streams in the series of reactors. An embodiment of the present disclosure is one, any or all of prior embodiments in this paragraph up through the first embodiment in this paragraph, wherein the second product stream is passed counter-currently to the first hydrocarbon feed stream and the second hydrocarbon feed stream in the heat exchanger.

A second embodiment of the present disclosure is a process for converting a feed in a series of reactors, comprising feeding a first hydrocarbon feed stream through tubes in a heat exchanger to provide a heat exchanged first hydrocarbon feed stream; charging the heat exchanged first hydrocarbon feed stream to a first reactor to provide a first product stream; feeding a second hydrocarbon feed stream through tubes in the heat exchanger to provide a heat exchanged second hydrocarbon feed stream; charging the heat exchanged second hydrocarbon feed stream to a second reactor to provide a second product stream; and feeding the second product stream to the shell of the heat exchanger to provide a heat exchanged a second product stream An embodiment of the present disclosure is one, any or all of prior embodiments in this paragraph up through the second embodiment in this paragraph, wherein the second product stream is at a higher temperature than the first hydrocarbon feed stream and the second hydrocarbon feed stream. An embodiment of the present disclosure is one, any or all of prior embodiments in this paragraph up through the second embodiment in this paragraph, wherein the first hydrocarbon feed stream is passed through the tubes of a first tube sheet and the second hydrocarbon feed stream is passed through the tubes of a second tube sheet in the heat exchanger. An embodiment of the present disclosure is one, any or all of prior embodiments in this paragraph up through the second embodiment in this paragraph, wherein the second product stream is passed counter-currently to the first hydrocarbon feed stream and the second hydrocarbon feed stream in the heat exchanger. An embodiment of the present disclosure is one, any or all of prior embodiments in this paragraph up through the second embodiment in this paragraph, wherein the heat exchanger is a spiral tube heat exchanger comprising at least two inlet tube sheets surrounded by the shell. An embodiment of the present disclosure is one, any or all of prior embodiments in this paragraph up through the second embodiment in this paragraph, wherein the series of reactors comprise four reactors. An embodiment of the present disclosure is one, any or all of prior embodiments in this paragraph up through the second embodiment in this paragraph, wherein the process comprises converting a naphtha stream to produce ethane and propane in the series of reactors.

A third embodiment of the present disclosure is a process for converting a feed in a series of reactors, comprising taking at least two hydrocarbon streams, a first hydrocarbon feed stream and a second hydrocarbon feed stream; heating the first hydrocarbon feed stream and the second hydrocarbon feed stream in a heat exchanger with a second product stream from a second reactor; and charging a heated first hydrocarbon feed stream into a first catalyst bed in a first reactor to produce a first product stream and a heated second hydrocarbon feed stream into a second catalyst bed in the second reactor to produce the second product stream. An embodiment of the present disclosure is one, any or all of prior embodiments in this paragraph up through the third embodiment in this paragraph, wherein the heat exchanger is a spiral tube heat exchanger comprising at least two inlet tube sheets surrounded by a shell.

Without further elaboration, it is believed that using the preceding description that one skilled in the art can utilize the present disclosure to its fullest extent and easily ascertain the essential characteristics of this disclosure, without departing from the spirit and scope thereof, to make various changes and modifications of the disclosure and to adapt it to various usages and conditions. The preceding preferred specific embodiments are, therefore, to be construed as merely illustrative, and not limiting the remainder of the disclosure in any way whatsoever, and that it is intended to cover various modifications and equivalent arrangements included within the scope of the appended claims.

In the foregoing, all temperatures are set forth in degrees Celsius and, all parts and percentages are by weight, unless otherwise indicated.

1. A process for converting a feed in a series of reactors, comprising:
heat exchanging a first hydrocarbon feed stream and a second hydrocarbon feed stream in a heat exchanger with a second product stream from a second reactor; and
charging a heat exchanged first hydrocarbon feed stream into a first catalyst bed in a first reactor to produce a first product stream and a heat exchanged second hydrocarbon feed stream into a second catalyst bed in the second reactor to produce the second product stream.

2. The process of claim 1, wherein the heat exchanger is a spiral tube heat exchanger comprising at least two inlet tube sheets surrounded by a shell.

3. The process of claim 2 further comprising:
passing said first hydrocarbon feed stream to a first tube sheet and passing said second hydrocarbon feed stream to a second tube sheet; and
passing said second product stream to a shell side of the heat exchanger to heat exchange with said first hydrocarbon feed stream and said second hydrocarbon feed stream to produce a heat exchanged second product stream.

4. The process of claim 1, wherein the heat exchanger is a plate heat exchanger.

5. The process of claim 1 further comprising a third reactor and comprising:
combining said first product stream from the first reactor with a third hydrocarbon feed stream to produce a combined third hydrocarbon feed stream;
heat exchanging said combined third hydrocarbon feed stream or said third hydrocarbon feed stream in the heat exchanger by heat exchanging with said second product stream from the second reactor; and
charging a heat exchanged combined third hydrocarbon feed stream to a third reactor to produce a third product stream.

6. The process of claim 5 further comprising a fourth reactor and comprising:
combining said third product stream from the third reactor with a fourth hydrocarbon feed stream to produce a combined fourth hydrocarbon feed stream;
heat exchanging said combined fourth hydrocarbon feed stream or said fourth hydrocarbon feed stream in the heat exchanger by heat exchanging with said second product stream from the second reactor; and
charging a heat exchanged combined fourth hydrocarbon feed stream to a fourth reactor to produce said fourth product stream.

7. The process of claim 6 further comprising:
combining said fourth product stream from the fourth reactor with said second hydrocarbon feed stream to produce a combined second hydrocarbon feed stream;
heat exchanging said combined second hydrocarbon feed stream or said second hydrocarbon feed stream in the heat exchanger by heat exchanging with said second product stream from the second reactor; and
charging a heat exchanged combined second hydrocarbon feed stream to the second reactor to produce said second product stream.

8. The process of claim 1, wherein said second product stream is at a higher temperature than said first hydrocarbon feed stream and said second hydrocarbon feed stream.

9. The process of claim 1, wherein the process comprises converting a naphtha stream to produce ethane and propane in the series of reactors.

10. The process of claim 1, wherein the process comprises oligomerizing said hydrocarbon streams in the series of reactors.

11. The process of claim 1, wherein said second product stream is passed counter-currently to said first hydrocarbon feed stream and said second hydrocarbon feed stream in the heat exchanger.

12. A process for converting a feed in a series of reactors, comprising:
feeding a first hydrocarbon feed stream through tubes in a heat exchanger to provide a heat exchanged first hydrocarbon feed stream;
charging the heat exchanged first hydrocarbon feed stream to a first reactor to provide a first product stream;
feeding a second hydrocarbon feed stream through tubes in said heat exchanger to provide a heat exchanged second hydrocarbon feed stream;
charging the heat exchanged second hydrocarbon feed stream to a second reactor to provide a second product stream; and
feeding said second product stream to the shell of said heat exchanger to provide a heat exchanged a second product stream.

13. The process of claim 12, wherein said second product stream is at a higher temperature than said first hydrocarbon feed stream and said second hydrocarbon feed stream.

14. The process of claim 12, wherein said first hydrocarbon feed stream is passed through the tubes of a first tube sheet and said second hydrocarbon feed stream is passed through the tubes of a second tube sheet in the heat exchanger.

15. The process of claim 12, wherein said second product stream is passed counter-currently to said first hydrocarbon feed stream and said second hydrocarbon feed stream in the heat exchanger.

16. The process of claim 12, wherein the heat exchanger is a spiral tube heat exchanger comprising at least two inlet tube sheets surrounded by the shell.

17. The process of claim 12, wherein the series of reactors comprise four reactors.

18. The process of claim 12, wherein the process comprises converting a naphtha stream to produce ethane and propane in the series of reactors.

19. A process for converting a feed in a series of reactors, comprising:
taking at least two hydrocarbon streams, a first hydrocarbon feed stream and a second hydrocarbon feed stream;
heating said first hydrocarbon feed stream and said second hydrocarbon feed stream in a heat exchanger with a second product stream from a second reactor; and
charging a heated first hydrocarbon feed stream into a first catalyst bed in a first reactor to produce a first product stream and a heated second hydrocarbon feed stream into a second catalyst bed in the second reactor to produce the second product stream.

20. The process of claim 19, wherein the heat exchanger is a spiral tube heat exchanger comprising at least two inlet tube sheets surrounded by a shell.
